(19) United States
(12) Patent Application Publication
(10) Pub. No.: US 2012/0283836 A1
(43) Pub. Date: Nov. 8, 2012

(54) INTERBODY FUSION DEVICE

(75) Inventor: Zhenqi Ding, Zhangzhou (CN)

(73) Assignees: THE 175TH HOSPITAL OF PLA, Fujian (CN); DING, Zhenqi, Fujian (CN)

(21) Appl. No.: 13/102,539

(22) Filed: May 6, 2011

(51) Int. Cl. A61F 2/44

(52) U.S. Cl. 623/17.16

(57) ABSTRACT

An interbody fusion device related to implants for orthopedic surgery has a hollow skeleton made of titanium alloy to hold nonstructural cancellous bone graft. The skeleton is a U-shaped plate, whose front wall is disposed with holes. The upper and the lower end of the bottom of the U-shaped plate have inclined holes of ladder shape. The bigger end of the inclined hole is placed on the bottom surface of the bottom of the U-shaped plate, and the smaller end of the inclined hole is placed on the corresponding surface of the bottom of the U-shaped plate; the inclined hole of the U-shaped plate is disposed with screw inside. The present invention has small volume and the vertebra is partial removal, so it is more reliable for interbody fusion.

### FIELD OF THE INVENTION

The present invention relates to an implant for orthopedic surgery.

### BACKGROUND OF THE INVENTION

To repair the damaged vertebrae, artificial vertebral body is come out in the medical community aboard, such as titanium cage (TMC). The titanium cage is a hollow skeleton made of titanium alloy formed by titanium alloy plate of several meshes and set in a circle, the inner of the titanium cage is disposed with ring-shaped support. When insert the TMC, the corpectomy or the total removal of the vertebral body needs to be done, and then pack the bone graft in it. The raised spots on the both ends of the titanium cage withstand the cephalad and the caudad endplates separately. With bone growth between vertebral body and bone graft inside the TMC, the fusion of the index level and adjacent vertebral body will be achieved. The minute vessels will grow with the bone graft through the meshes of the titanium cage, achieving the treatment of interbody fusion.

But the titanium cage has these disadvantages below:

1). To implant the bulky size of cylindrical titanium cage, corpectomy or the total removal of the damaged vertebral body needs to be done, the patient will blood much and it will damage the blood supply to the vertebrae.
2). The titanium cage is fixed to the vertebrae by the raised spots on the both ends of the titanium cage withstanding the cephalad and the caudad endplates separately, which will be easily caved in.
3). Bone grafting could not be took in the surrounding of the titanium cage, and the titanium cage does not have the effect of damper to keep out the vertebrae canal, so that the bone graft could enter in and compress the nerve structure.
4). The titanium cage would encase the implant bone, keep off the X-ray and prevent from observing the growth of the bone.

### SUMMARY OF THE INVENTION

The object of the present invention is to provide interbody fusion device, which could prevent the disadvantages of the titanium cage.

The present invention provides interbody fusion device, which is a hollow skeleton made of titanium alloy for holding bone graft, wherein the skeleton is a U-shaped plate, the front wall is equipped with several holes while the back wall has not; the upper end and the lower end of the bottom of the U-shaped plate are configured separately with inclined hole of ladder shaped, the bigger end of the inclined hole is placed on the bottom surface of the bottom of the U-shaped plate, and the smaller end of the inclined hole is placed on the corresponding surface of the bottom of the U-shaped plate; the inclined hole of the U-shaped plate is disposed with screw inside.

The outward of the cap of the two screws are circle-arc surface, the boundary between the bigger end and the smaller end of the inclined holes is circle arc section, which is coupled to the circle-arc surface.

The outer surface of the back wall of the U-shaped plate is configured with slope along the free end; the outer surface of the front wall is configured with slope along the free end. The slope provides an insert guide head for the U-shaped plate in the surgery.

The upper ends and lower ends of the back wall, front wall and the bottom of the U-shaped plate are configured with flanges. The flanges produce friction between the U-shaped plate and the caudad endplate of the upper vertebral body and the cephalad endplate of the lower one, which improves the stability of the U-shaped plate.

The upper and the lower ends of the U-shaped plate is configured parallel to each other or not. The upper and the lower ends of the U-shaped plate parallel to each other makes it more fit to the thoracic vertebrae end-plate; the upper and the lower ends of the U-shaped plate unparallel to each other makes it more fit to the lumbar vertebrae end-plate; they could prevent collapse or fracture of the upper and the lower lumbar vertebrae due to the partial pressure.

The present invention of interbody fusion device (it could be also called U-shaped artificial vertebrae) comprises a titanium alloy U-shaped plate of 0.6 cm and two screws locking the U-shaped plate between the caudad endplate of the upper vertebral body and the cephalad endplate of the lower one. The inner and the front of the U-shaped plate could be implanted with large amount of bone graft, making it easily to form biological connection. Corpectomy or the total removal of the damaged vertebral body is not needed and only one-third of posterior part of vertebral body is removed because of the small volume of the U-shaped plate. It keeps the anterior column and part of middle column of the damaged vertebral body. It retains most of the vertebrae bone, which reduces blooding and supports the vertebrae blood supply, beneficial to the interbody fusion of the anterior-middle column of the damaged vertebrae. The back wall of the U-shaped plate serving as a gate is disposed without holes, which prevent the entry of the bone graft to the vertebrae canal and the compression to the nerve structures. The front wall of the U-shaped plate is disposed with holes, making it easier for the biological connecting. The minute vessel grows accompanying with the bone by the holes. The inclined holes at the upper and the lower end of the bottom of the U-shaped plate are disposed with screws inside, the screws lock to the caudad endplate of the upper vertebral body and the cephalad endplate of the lower one, preventing the collapse of the implant. The interbody fusion situation could be direct evaluated by the observing the healing of the front two-third vertebrae when taking imaging examination.

### BRIEF DESCRIPTION OF THE DRAWINGS

FIG. 1 illustrates the structure of the first embodiment of the present invention;

FIG. 2 illustrates the structure of the U-shaped plate of the first embodiment;

FIG. 3 illustrates the upward view of the U-shaped plate of the first embodiment;

FIG. 4 illustrates the cross-section view of the U-shaped plate of the first embodiment;

FIG. 5 illustrates the structure of the screw of the first embodiment;

FIG. 6 illustrates the status of used in the first embodiment;

FIG. 7 illustrates the structure of the second embodiment of the present invention;

FIG. 8 illustrates the U-shaped plate of the second embodiment;

FIG. 9 illustrates the upward view of the U-shaped plate of the second embodiment;

FIG. 10 illustrates the sectional structure of the U-shaped plate of the second embodiment;

FIG. 11 illustrates the status of used in the second embodiment.

### DETAILED DESCRIPTION OF THE INVENTION

### The First Embodiment

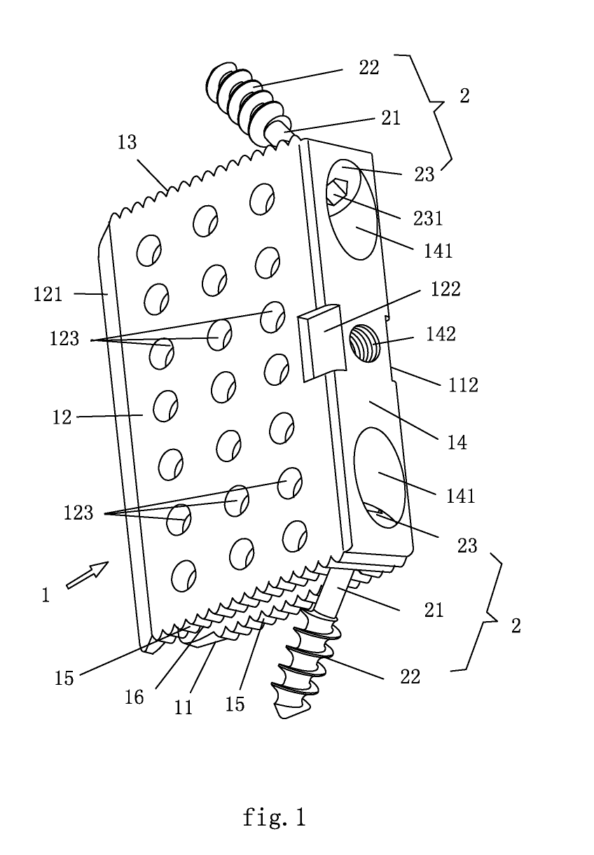

The object of the present invention is to provide a preferred embodiment of fusion device for vertebral body. The FIG. 1 illustrates the structure of the first embodiment; it comprises a U-shaped plate 1 and two screws 2.

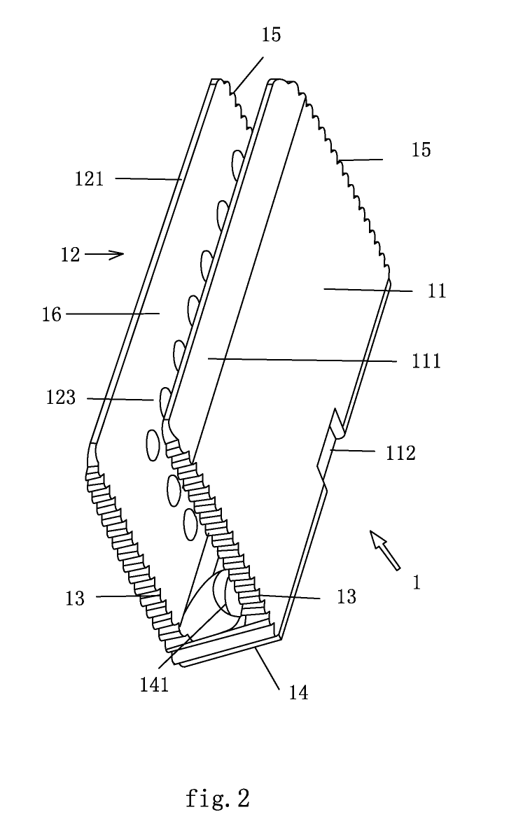

The U-shaped plate 1 is hollow skeleton made of titanium alloys with thickness of 0.6 cm. FIG. 2 illustrates the structure of the U-shaped plate. The back wall 11 of the U-shaped plate 1 connects to the front wall 12 by the bottom 14. The upper end and the lower end of the U-shaped plate 1 are disposed unparallel to each other and configured with included angle of slope. The upper ends of the back wall 11, front wall 12 and the bottom 14 are configured with flanges 13. The lower ends of the back wall 11, the front wall 12 and the bottom 14 are configured with flanges 15. The back wall 11, the front wall 12 and the bottom 14 are circling around forming the inside groove 16.

The outer surface of the back wall 11 of the U-shaped plate 1 is configured with slope 111 along the free end. The middle section of the back wall 11 near the bottom 14 is configured with a back groove 112 extending to the edge.

Refer to the FIG. 1: The outer surface of the front wall 12 of the U-shaped plate 1 is configured with slope 121 along the free end. The middle section of the front wall 12 near the bottom 14 is configured with a front groove 122 extending to the edge. Several holes 123 are disposed on the front wall 12 evenly disposed.

The slope 111 on the back wall 11 and the slope 121 on the front wall 12 of the U-shaped plate 1 provide a leading section for the placement of the U-shaped plate in the surgery. The back groove 112 on the back wall 11 and the front groove 122 on the front wall 12 of the U-shaped plate provide a gripper for holding the U-shaped plate in the surgery.

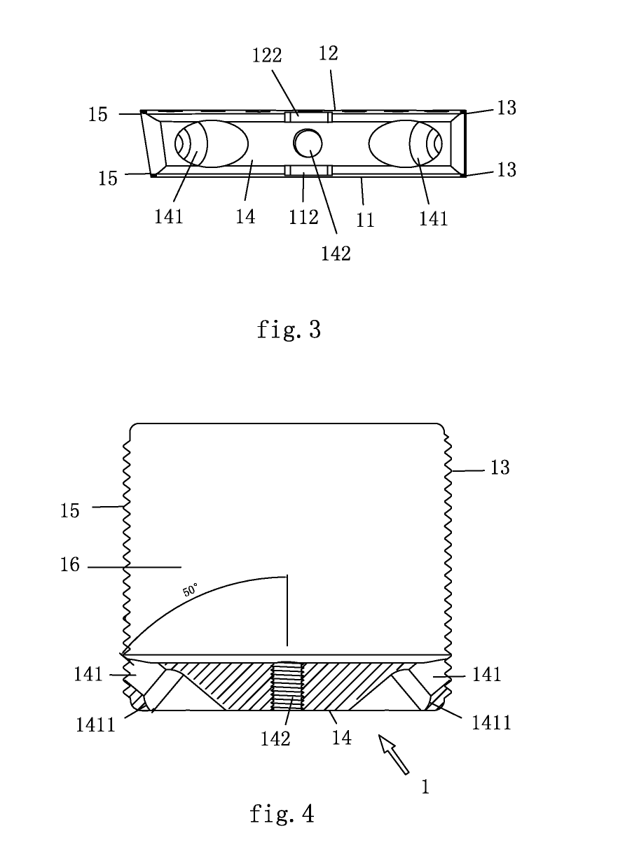

Refer to the FIG. 3: the upper and the lower ends of the bottom 14 are disposed with inclined hole 141 of ladder shaped separately; the angle of the axis of the inclined hole 141 with the normals of the bottom 14 is 50 degrees. The bigger end of the two inclined holes 141 is disposed on the bottom surface of the bottom 14 and the smaller end on the corresponding surface of the bottom 14. The boundary between the bigger end and the smaller end of the inclined hole 141 is circle- arc section 1411. The middle of the bottom 14 is disposed with screw hole 142, to fix an assistant potable bar to put the U-shaped plate 1 to the opening position of the damaged vertebrae.

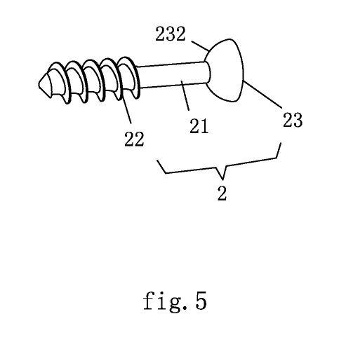

The screw 2 is made of titanium alloy. FIG. 5 illustrates the structure of the screw 2. The forward part of the screw body 21 is configured with cone thread section 22, which could be easily screwed to the bone and have good performance of anti-pull out. The backend of the screw body 21 is disposed with cap 23. Refer to FIG. 1; the end face of the cap 23 has hexagon jack 231, adapting to the hexagon screw-driver of high safety in the surgery. The outward of the cap 23 is circle arc surface 232, which is coupled to the circle-arc section 1411 of the inclined hole 141.

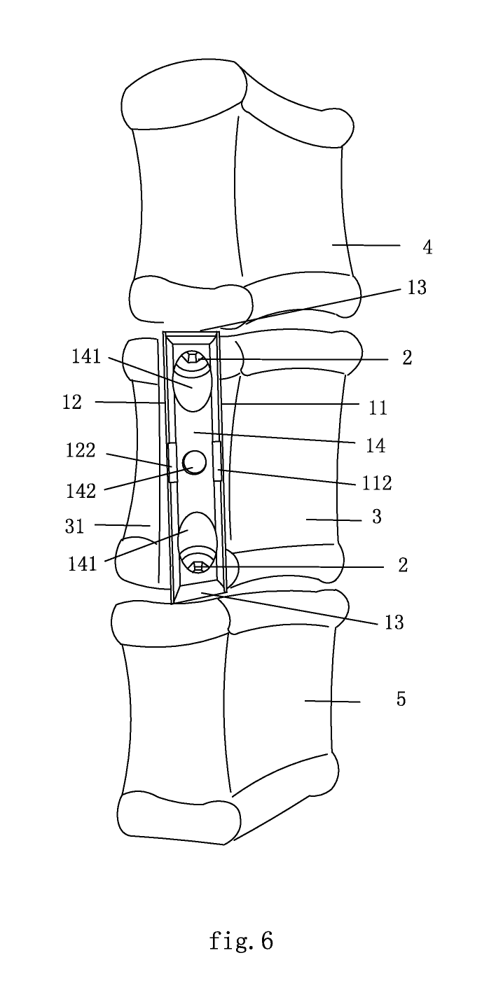

Refer to the FIG. 6, the usage status of the first embodiment of the interbody fusion device. The rear edge of the damaged lumbar vertebrae 3 is disposed with a longitudinal groove to contain the U-shaped plate. Retain the anterior column 31 and part of the middle column of the damaged lumbar vertebrae. Put the bone graft into the inner groove 16 of the U-shaped plate, observe the bone graft filling status through the holes 123 on the front wall 12 and adjust the filler to make the inner groove 16 of the U-shaped plate filling with bone graft. Hold the back groove 112 on the back wall 11 and the front groove 122 of the front wall 12 with gripper; put the free ends of the back wall 11 and the front wall 12 towards the longitudinal groove of the damaged lumbar vertebrae 3. The front wall 12 goes towards the anterior column 31. And the front of the front wall 12 can be implanted with bone for biological connecting.

The back wall 11 of the U-shaped plate 1 goes towards the vertebrae canal, preventing the entry of the bone graft to the vertebrae canal. If the bone graft gets into the vertebrae canal, nerve compression would occur. The flanges 13 and flanges 15 on the both ends of the u-shaped plate 1 withstand the caudad endplate of the upper vertebral body 4 and the cephalad endplate of the lower vertebral body 5 separately. Two screws 2 pass though the corresponding inclined holes 141 on the both end of the bottom of the U-shaped plate 1, and the two screws 2 reach out of the both ends of the bottom. The cone thread sections 22 of the two screws 2 lock the bottom of the cephalad lumbar vertebrae 4 and the top of the caudad lumbar vertebrae 5, and the circle arc surface 232 on the cap 23 of the screw 2 contact with the circle- arc section 1411 of the inclined hole 141. The U-shaped plate 1 is fixed between the cephalad lumbar vertebrae 4 and the caudad lumbar vertebrae 5 by the two screws 2.

### The Second Embodiment

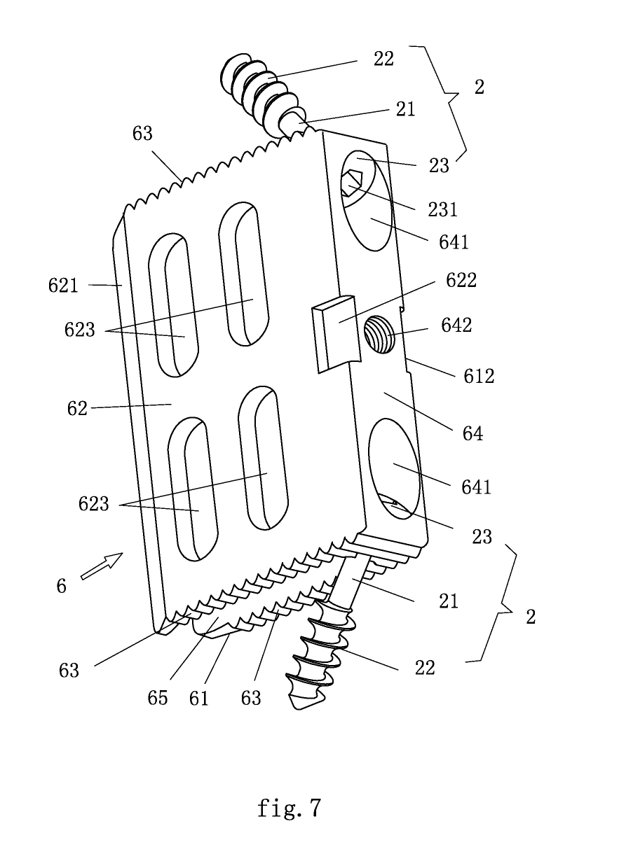

The present invention provides with the second embodiment of the interbody fusion device for the thoracic vertebral body. FIG. 7 illustrates the structure of the second embodiment, which comprises of a U-shaped plate 6 and two screws 2. The structure of the screws 2 is the same with that of the first embodiment.

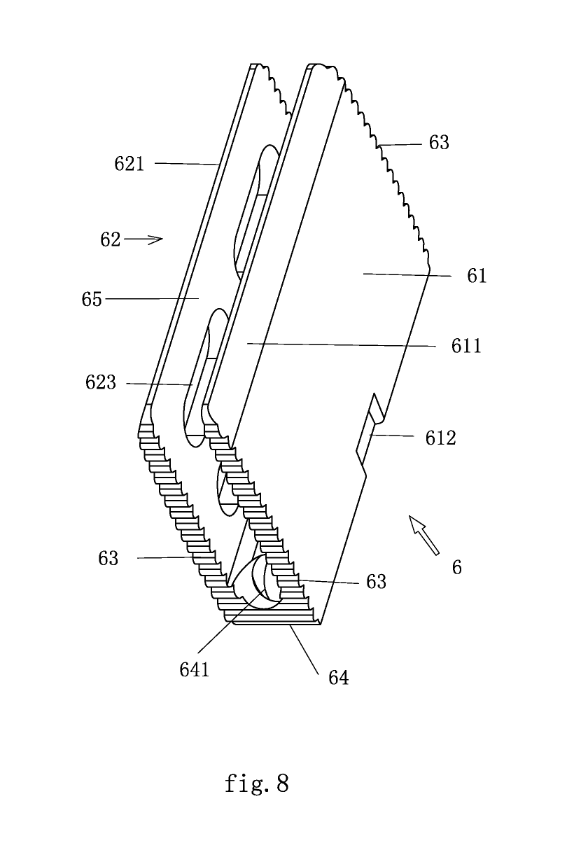

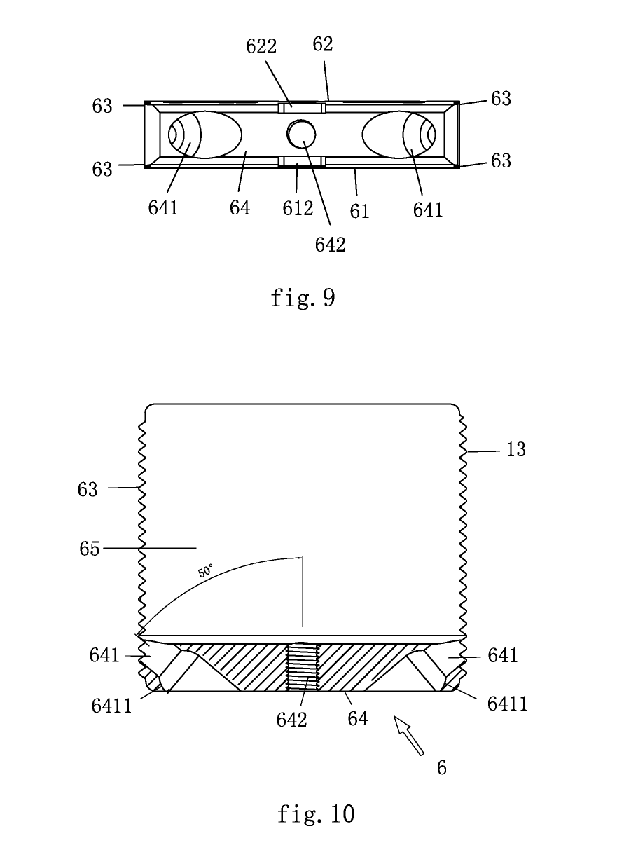

The U-shaped plate 6 is hollow skeleton made of titanium alloys with thickness of 0.6 cm. FIG. 8 illustrates the structure of the U-shaped plate. The back wall 61 of the U-shaped plate 6 connects to the front wall 62 by the bottom 64. The upper end and the lower end of the U-shaped plate 6 are disposed parallel to each other and configured with included angle of slope, which makes it more fit to the thoracic vertebrae end-plate. The upper ends of the back wall 61, front wall 62 and the bottom 64 are configured with flanges 63. The back wall 61, the front wall 62 and the bottom 64 are circling around forming the inside groove 65.

The outer surface of the back wall 61 of the U-shaped plate 6 is configured with slope 611 along the free end. The middle section of the back wall 61 near the bottom 64 is configured with a back groove 612 extending to the edge.

The outer surface of the front wall 62 of the U-shaped plate 6 is configured with slope 621 along the free end. The middle section of the front wall 62 near the bottom 64 is configured with a front groove 622 extending to the edge. Four holes 623 are disposed on the front wall 62 evenly disposed.

The slope 611 on the back wall 61 and the slope 621 on the front wall 62 of the U-shaped plate 6 provide a leading section for the placement of the U-shaped plate in the surgery. The back groove 612 on the back wall 11 and the front groove 622 on the front wall 62 of the U-shaped plate provide a gripper for holding the U-shaped plate in the surgery.

Refer to the FIG. 10: the upper and the lower ends of the bottom 64 are disposed with inclined hole 641 of ladder shaped separately; the angle of the axis of the inclined hole 641 with the normals of the bottom 64 is 50 degrees. The bigger end of the two inclined holes 641 is disposed on the bottom surface of the bottom 64 and the smaller end on the corresponding surface of the bottom 64. The boundary between the bigger end and the smaller end of the inclined hole 641 is circle- arc section 6411. The circle-arc section 6411 is coupled to the circle-arc surface 232 on the outward of the cap 23 of the screws 2. The middle of the bottom 64 is disposed with screw hole 642, to fix an assistant potable bar to put the U-shaped plate 6 to the opening position of the damaged thoracic vertebrae.

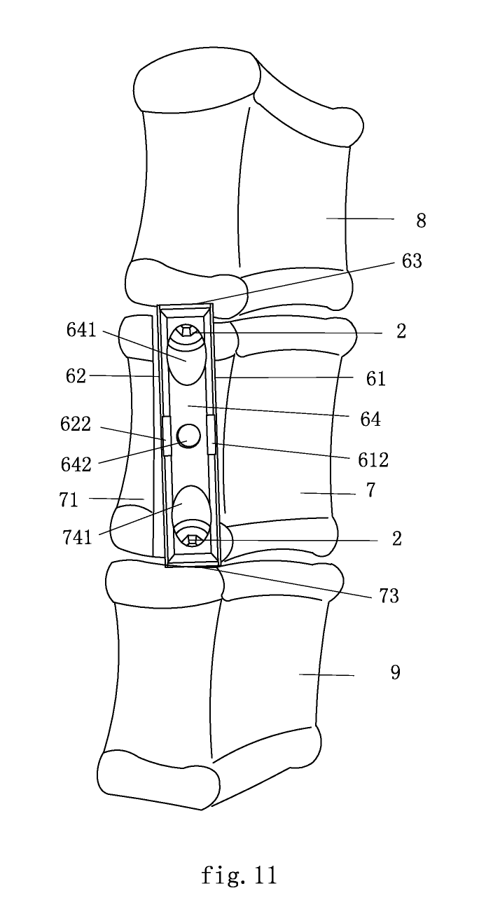

Refer to the FIG. 11, the usage status of the second embodiment of the interbody fusion device. The rear edge of the damaged thoracic vertebrae 7 is disposed with a longitudinal groove to contain the U-shaped plate 6. Retain the anterior column 71 and part of the middle column of the damaged thoracic vertebrae. Put the bone graft into the inner groove 65 of the U-shaped plate, observe the bone graft filling status through the holes 623 on the front wall 62 and adjust the filler to make the inner groove 65 of the U-shaped plate full with bone graft. Hold the back groove 612 on the back wall 61 and the front groove 622 of the front wall 62 with gripper; put the free ends of the back wall 61 and the front wall 62 towards the longitudinal groove of the damaged thoracic vertebrae 7. The front wall 62 goes towards the anterior column 71. And the front of the front wall 62 can be implanted with bone for biological connecting.

The back wall 61 of the U-shaped plate 6 goes towards the vertebrae canal, and the back wall 61 can prevent the entry of the bone graft to the vertebrae canal. If the bone graft gets into the vertebrae canal, nerve compression would occur. The flanges 63 on the both ends of the u-shaped plate 6 withstand the caudad endplate of the upper vertebral body 8 and the cephalad endplate of the lower vertebral body 9 separately. Two screws 2 pass though the corresponding inclined holes 641 on the both end of the bottom of the U-shaped plate 6, and the two screws 2 reach out of the both ends of the bottom. The cone thread sections 22 of the two screws 2 lock the bottom of the cephalad thoracic vertebrae 8 and the top of the caudad thoracic vertebrae 9, and the circle arc surface 232 on the cap 23 of the screw 2 contact with the circle-arc section 6411 of the inclined hole 641. The U-shaped plate 6 is fixed between the cephalad thoracic vertebrae 8 and the caudad thoracic vertebrae 3 by the two screws 2.

Although the present invention has been fully described by ways of preferred embodiments with reference to the accompanying drawings, it is to be noted that various changes and modifications will be apparent to those skilled in the art. Therefore, unless otherwise such changes and modifications depart from the scopes of the present invention, they should be construed as being included therein.

What is claimed is:

1. Interbody fusion device, which is a hollow skeleton made of titanium alloy for holding bone graft, wherein the skeleton is a U-shaped plate, the front wall is equipped with several holes while the back wall has not; the upper end and the lower end of the bottom of the U-shaped plate are configured separately with inclined hole of ladder shaped, the bigger end of the inclined hole is placed on the bottom surface of the bottom of the U-shaped plate, and the smaller end of the inclined hole is placed on the corresponding surface of the bottom of the U-shaped plate; the inclined hole of the U-shaped plate is disposed with screw inside.

2. Interbody fusion device according to the claim 1, wherein the outward of the cap of the two screws are circle-arc surface, the boundary between the bigger end and the smaller end of the inclined holes is circle arc section, which is coupled to the circle-arc surface.

3. Interbody fusion device according to the claim 1, wherein the outer surface of the back wall of the U-shaped plate is configured with slope along the free end, the outer surface of the front wall is configured with slope along the free end.

4. Interbody fusion device according to the claim 1, wherein the upper ends and lower ends of the back wall, front wall and the bottom of the U-shaped plate are configured with flanges.

5. Interbody fusion device according to the claim 1, wherein the upper and the lower ends of the U-shaped plate are disposed parallel to each other or not.

6. Interbody fusion device according to the claim 2, wherein the outer surface of the back wall of the U-shaped plate is configured with slope along the free end, the outer surface of the front wall is configured with slope along the free end.

7. Interbody fusion device according to the claim 2, wherein the upper ends and lower ends of the back wall, front wall and the bottom of the U-shaped plate are configured with flanges.

8. Interbody fusion device according to the claim 2, wherein the upper and the lower ends of the U-shaped plate are disposed parallel to each other or not.
